(19) United States
(12) Patent Application Publication
(10) Pub. No.: US 2025/0022609 A1
(43) Pub. Date: Jan. 16, 2025

(54) PATIENT POOLING BASED ON MACHINE LEARNING MODEL

(71) Applicant: Roche Molecular Systems, Inc., Pleasanton, CA (US)

(72) Inventors: Fernando Garcia-Alcalde, Basel (CH); Elspeth Moira Fraser Horne, Edinburgh (GB); Carsten Magnus, Zurich (CH); Athanasios Siadimas, Basel (CH); Antoaneta Petkova Vladimirova, Mountain View, CA (US)

(73) Assignee: Roche Molecular Systems, Inc., Pleasanton, CA (US)

(21) Appl. No.: 18/902,082

(22) Filed: Sep. 30, 2024

Related U.S. Application Data
(63) Continuation of application No. PCT/EP2023/058428, filed on Mar. 31, 2023.
(60) Provisional application No. 63/362,373, filed on Apr. 1, 2022.

(51) Int. Cl. G16H 50/30; G16H 10/40; G16H 10/60; G16H 20/10; G16H 50/70

(52) U.S. Cl. CPC G16H 50/30; G16H 10/40; G16H 10/60; G16H 20/10; G16H 50/70

(57) ABSTRACT

Disclosed herein are techniques for facilitating a clinical decision for a patient based on identifying a group of patients having similar attributes as the patient. The group of patients can be identified using information from a predictive machine learning model that performs a clinical prediction for the patient. At least some of the attributes of the group of patients can be output to support a clinical decision. The attributes may include, for example, biography data of the patient, results of one or more laboratory tests of the patient, biopsy image data of the patient, molecular biomarkers of the patient, a tumor site of the patient, and a tumor stage of the patient.

### CROSS-REFERENCE TO RELATED APPLICATIONS

This application is a continuation of International Patent Application No. PCT/EP2023/058428, filed Mar. 31, 3023, which claims priority to U.S. Patent Application No. 63/362,373, filed Apr. 1, 2022, the disclosures of which are hereby incorporated by reference in its entirety.

### BACKGROUND

Predictive machine learning models trained using real world clinical data offer tremendous potential to provide patients and their clinicians patient-specific information regarding diagnosis, prognosis or optimal therapeutic course. The machine learning models can be trained to perform a clinical prediction to predict a medical outcome for a patient such as, for example, a probability of survival of the patient as a function of time from a diagnosis (e.g., an advanced stage cancer), a survival time of the new patient from the diagnosis, other types of prognosis, etc. The prediction can be provided to the patient to, for example, improve the patient's ability to plan for his/her future, which can improve the quality of life of the patient.

Many machine learning models are developed utilizing a broad spectrum of data for many types of patients with differing conditions, treatment modalities applied to those conditions, and prognosis. Such models may not be trained sufficiently with data applicable to a particular patient type and/or may be weighted from data that may be of low value to a particular patient type, leading to inaccurate prognosis and/or recommended treatments. Thus, improved methods of utilizing machine learning models for enhancing patient care are needed.

### BRIEF SUMMARY

Disclosed herein are techniques for facilitating a clinical decision for a patient based on identifying a group of patients having similar attributes as the patient. The group of patients can be identified using information from a predictive machine learning model that performs a clinical prediction for the patient. At least some of the attributes of the group of patients can be output to support a clinical decision. The attributes may include, for example, biography data of the patient, results of one or more laboratory tests of the patient, biopsy image data of the patient, molecular biomarkers of the patient, a tumor site of the patient, and a tumor stage of the patient.

Specifically, a clinical decision support system can employ a machine learning model to make a clinical prediction for a patient based on the attributes of the patient. For example, the machine learning model can include a random survival forest (RSF) model to predict a probability of survival of the patient as a function of time elapsed from diagnosis. The clinical decision support system can also identify a group of patients (e.g. a “similarity-based patient pool”) having certain attributes that are similar to those of the patient. The similarity-based patient pool can include patients having comparable health conditions to the patient and can be identified based on these patients being similar to the patient in a subset of the attributes that are most relevant to the clinical prediction performed by the machine learning model (e.g., the probability of survival at a particular time point from diagnosis). The clinical decision support system can then obtain information of the attributes of the similarity-based patient pool.

A clinical decision support system can output a predicted probability of survival for the new patient, as well as the new patient's attributes that are determined to be most relevant to this prediction. With similarity-based patient pooling, the clinical decision support system can output the survival function for the similarity-based patient pool, as well as a summary of the attributes of the patients in the similarity-based patient pool. This facilitates a comparison between the attributes of the new patient and the attributes of the similarity-based patient pool, focusing on the attributes that are most relevant to the survival prediction for the new patient. Investigating the relationship between attributes and survival in the similarity-based patient pool may help the clinician to determine courses of action (e.g. treatments) to improve the probability of survival for the new patient.

In some embodiments, a computer-implemented method of facilitating a clinical decision includes receiving first data corresponding to a plurality of features of a first patient, each feature representing an attribute of a plurality of attributes; inputting the first data to a machine learning model to generate a result of a clinical prediction for the first patient, the machine learning model being associated with a plurality of feature importance metrics, the plurality of feature importance metrics defining a relevance of each of the plurality of features to the clinical prediction; obtaining second data corresponding to the plurality of features of each of a group of patients based on a degree of similarity in at least some of the plurality of features between the first patient and the group of patients, the degree of similarity being based on the first data, the second data, and the plurality of feature data importance metrics; generating content based on the result of the clinical prediction and at least a part of the second data; and outputting the content to enable a clinical decision to be made for the first patient based on the content.

In some embodiments, the plurality of attributes includes at least one of: biography data of a patient, results of one or more laboratory tests of the first patient, biopsy image data of the first patient, molecular biomarkers of the first patient, a tumor site of the first patient, or a tumor stage of the first patient.

In some embodiments, the clinical prediction includes at least one of: a probability of survival of the first patient at a pre-determined time from when the first patient is diagnosed of having a tumor, a survival time of the first patient from when the first patient is diagnosed of having the tumor, or an outcome of receiving a treatment.

In some embodiments, the machine learning model includes a random forest survival model, the random forest survival model comprising a f decision trees each configured to process a subset of the first subset of the data to generate a cumulative survival probability; and wherein the survival rate of the patient at the pre-determined time is determined based on an average of the cumulative survival probabilities output by the plurality of decision trees.

In some embodiments, the group of patients is a first group of patients; wherein the first group of patients is selected from a second group of patients; and wherein the machine learning model is trained based on patient data of the second group of patients.

In some embodiments, the method further includes ranking the plurality of features based on the relevance of each feature of the plurality of features to the clinical prediction; determining a subset of the plurality of features based on the ranking; and determining the first group of patients based on the degree of similarity in the subset of the plurality of features between the first patient and the first group of patients.

In some embodiments, the first group of patients is selected from the second group of patients based on the degree of similarity in the subset of the plurality of features between the first patient and the first group of patients exceeding a threshold.

In some embodiments, the first group of patients is selected from the second group of patients based on selecting a threshold number of patients having the highest degree of similarity in the subset of the plurality of features with the first patient.

In some embodiments, the method further includes computing a weighted aggregated degree of similarity based on summing a scaled degree of similarity in each feature of the at least some of the plurality of features, each degree of similarity being scaled by a weight based on the relevance of the feature; and identifying the group of patients based on the weighted aggregated degree of similarities between the first patient and each of the group of patients.

In some embodiments, the feature importance metric of a feature is determined based on a relationship between errors of the results of clinical prediction generated by the machine learning model for a second patient of the first group of patients; wherein the results of clinical prediction are generated from a plurality of values of the feature of the second patient; and wherein the errors are computed based on comparing the results of the clinical prediction and an actual clinical outcome of the second patient.

In some embodiments, the content includes at least one of: a median survival time of the first group of patients, or a Kaplan-Meier survival curve of the first group of patients.

In some embodiments, the content includes values of one or more of the first subset of the plurality of features of the first patient, the first group of patients, and the second group of patients.

In some embodiments, a computer product includes a computer readable medium storing a plurality of instructions for controlling a computer system to perform an operation of any of the methods above.

In some embodiments, a system includes the computer product described herein; and one or more processors for executing instructions stored on the computer readable medium.

In some embodiments, a system includes means for performing any of the methods described herein.

In some embodiments, a system is configured to perform any of the methods described herein.

In some embodiments, a system includes modules that respectively perform the steps of any of the methods described herein.

These and other exemplary embodiments are described in detail below. For example, other embodiments are directed to systems, devices, and computer readable media associated with methods described herein.

A better understanding of the nature and advantages of examples of the present disclosure may be gained with reference to the following detailed description and the accompanying drawings.

### BRIEF DESCRIPTION OF THE DRAWINGS

The detailed description is set forth with reference to the accompanying figures.

FIG. 1A, FIG. 1B, and FIG. 1C illustrate example techniques for facilitating a clinical decision based on a clinical prediction, according to certain aspects of this disclosure.

FIG. 2A, FIG. 2B, FIG. 2C, FIG. 2D, FIG. 2E, and FIG. 2F illustrate an improved clinical decision system that enables machine-learning based patient pooling, according to certain aspects of this disclosure.

FIG. 3 illustrates a method of performing a machine-learning based patient pooling operation, according to certain aspects of this disclosure.

FIG. 4 illustrates an example computer system that may be utilized to implement techniques disclosed herein.

FIG. 5 illustrates an example of how the patient data from a patient pool can be used.

FIG. 6 illustrates another example of how the patient data from a patient pool can be used.

### DETAILED DESCRIPTION

As described above, a predictive machine learning model can be trained to perform a clinical prediction to predict a medical outcome for a new patient. The new patient can be any patient who is alive and the one for whom clinical decisions are being made. For example, a random survival forest (RSF) model can be trained based on the data of previous patients, as well as their survival statistics, to predict a probability of survival for a new patient as a function of time from a diagnosis (e.g., of an advanced stage cancer). The prediction can be provided to the new patient to, for example, improve their ability to plan for the future. This has the potential to improve the patient's quality of life.

Although a clinical prediction provided by a predictive machine learning model can provide valuable information to the new patient, the clinical prediction result by itself may not provide insight into how to improve the prognosis of the new patient. For example, a prediction that a patient has a certain likelihood of survival at a certain time point may not provide information about potential clinical decisions to improve the patient's likelihood of survival at that time point.

On the other hand, medical journeys of previous patients, whose data and survival statistics are used to train the predictive machine learning model, can provide valuable insights into potential clinical decisions to improve the prognosis of the new patient. For example, a machine learning model, such as an RSF model, can output a prediction of the probability that the new patient will survive until a certain time-point from diagnosis. There may exist a first group of patients (e.g., group A) whose survival probabilities with respect to time are similar to those predicted for the new patient by the model, and a second group of patients (e.g., group B) whose survival probabilities are far lower than those predicted for the new patient by the model. If group A shares a common biomarker with the new patient, while group B does not have that biomarker, it may be determined that the biomarker may be relevant to the new patient's probability of survival. A treatment decision can then be made to target the biomarker. But as described above, while a predictive machine learning model can be useful in predicting a prognosis for a patient based on the patient's attributes, a machine learning model typically does not identify other groups of patients whose medical outcomes are similar to the prognosis of the patient. Besides providing the clinical prediction result, the machine learning model typically does not provide additional information that can be used to improve the prognosis of the patient.

Disclosed herein are techniques for facilitating a clinical decision for a new patient based on identifying a group of patients (hereinafter, “similarity-based patient pool”) having similar attributes as the new patient. A predictive machine learning model is provided to perform a clinical prediction for the new patient, who is alive and whose future survival is unknown. The similarity-based patient pool can be identified from a group of previous patients whose data and survival statistics are used to train the predictive machine learning model. At least some of the attributes of the similarity-based patient pool can be output to support a clinical decision. The attributes may include, for example, biography data of the patient, results of one or more laboratory tests of the patient, biopsy image data of the patient, molecular biomarkers of the patient, a tumor site of the patient, and a tumor stage of the patient.

In some examples, a clinical decision support system can employ a machine learning model to perform a clinical prediction for a new patient based on the attributes of the new patient. For example, a random survival forest (RSF) model can be used to predict a probability of survival of the patient as a function of time elapsed from diagnosis. The clinical decision support system can also identify a similarity-based patient pool with certain attributes that are similar to those of the new patient. The similarity-based patient pool can be identified based on patients sharing similar values to the new patient in a subset of the attributes that are determined to be most relevant to the clinical prediction performed by the machine learning model (e.g., the probability of survival at a particular time-point from diagnosis). The clinical decision support system can output the attributes and the medical outcomes of the similarity-based patient pool, along with the attributes and the clinical prediction result of the patient, to facilitate a clinical decision for the patient. In some examples, the similarity-based patient pool can include patients whose attributes and survival rate statistics are included in the training data to train the machine learning model. In some examples, the similarity-based patient pool can also include patients whose data are not used to train the machine learning model.

Specifically, the clinical decision support system can receive first data corresponding to the attributes of the new patient. The attributes can include various biographical information, such as age and gender of the patient. Each attribute can be represented as a feature, which can include one or more vectors for input into the machine learning model. In some examples, an attribute can be represented by multiple features. The attributes may also include a history of the patient (e.g., which treatment(s) the patient has received), a habit of the patient (e.g., whether the patient smokes), categories of laboratory test results of the patient (e.g. leukocytes count, a hemoglobin count, a platelets count, a hematocrit count, an erythrocyte count, a creatinine count, a lymphocytes count, measurements of protein, bilirubin, calcium, sodium, potassium, glucose). The attributes may also indicate measurements of various biomarkers for different cancer types, such as oestrogen receptor (ER), progesterone receptor (PR), human epidermal growth factor receptor 2 (HER2), epidermal growth factor receptor (EGFR, or HER1) for breast cancer, ALK (anaplastic lymphoma kinase) for lung cancer, KRAS gene for lung and colorectal cancers, BRAF gene for colorectal cancer, etc. The attributes data can be processed by the clinical decision support system, or processed prior to input to the clinical decision support system, to create a plurality of features that contain the attribute information in a format (e.g., vectors) that can be interpreted by a machine learning model.

The clinical decision support system can include a machine learning model, which can be trained based on data from previous patients, to perform the clinical prediction for the new patient. The prediction can be based on inputting the attributes of the new patient to the machine learning model. For example, the machine learning model may include a RSF model that can output, as the clinical prediction, a predicted survival function, based on the first data. The survival function can be used to obtain the probability that the new patient survives until a pre-determined time (e.g., 500 days, 1000 days, 1500 days, etc.) after the new patient is diagnosed of a medical condition (e.g., an advanced stage cancer). Alternatively, a hazard function provides the risk of death as a function of time, given survival up until that time. Another example of the survival function is a cumulative hazard function (CHF) which provides an accumulation of the risk as a function of time. The survival function with respect to time can be used to generate a patient-specific survival plot for the new patient.

As part of the training operation, a plurality of feature importance metrics associated with the machine learning model can also be obtained, with the feature importance metrics defining the relevance of each feature to the clinical prediction (e.g., a survival rate at a particular time point). In one example, out-of-bag (OOB) samples, which include samples of the training patient data not used in building the RSF model, can be input to the decision trees to compute a prediction error, such as a concordance index (c-index). For those samples, the values for a feature can then be permuted, and the prediction errors for each decision tree can be calculated for the permuted values of that feature. A raw importance score for that feature can be computed based on averaging differences in the prediction errors among the trees for the permuted values. A higher raw importance score can indicate that the feature is more relevant to the predicted survival function, whereas a lower raw importance score can indicate that the feature is less relevant to the predicted survival function. At the end of the training operations, the features can be ranked based on their importance scores, with more relevant feature ranked higher.

Based on the new patient's attributes, the clinical decision support system can identify a group of patients from the patient database who are similar to the new patient in the highest-ranked features. This group can be referred to as the similarity-based patient pool. The first step in selecting patients to form the similarity-based patient pool is to calculate the similarity between the new patient and each of the patients in the database, based on the highest-ranked features. The patients who form the similarity-based patient pool are then selected based on a criterion, two examples of which are as follows. In the first example, the patients can be selected based on their similarity to the new patient exceeding a threshold. In the second example, patients in the database are ranked according to their similarity to the new patient, and a pre-determined number of patients with the highest rank are selected. Therefore, the similarity-based patient pool may be considered to not only have similar health conditions as the patient, but also being similar to the patient in the features that are most relevant to the clinical prediction.

The clinical decision support system can then output the attributes and the medical outcomes of the similarity-based patient pool, as well as the attributes and the clinical prediction result of the new patient. This may help to facilitate a clinical decision for the new patient. For example, the clinical decision support system can output a summary of the attributes of the similarity-based patient pool, along with a comparison of the attributes (especially those corresponding to the highest-ranked features) between the new patient and the similarity-based patient pool. The outputs of the clinical decision support allow a clinician to investigate the relevant attributes and to determine courses of actions (e.g., treatments) to improve the probability of survival of the new patient.

As an illustrative example, the feature corresponding to a biomarker attribute (e.g., epidermal growth factor receptor (EGFR)) may be one of the highest-ranked features for the RSF model. Suppose the new patient is EGFR-positive, and the clinical decision support system can output the EGFR positivity results for the similarity-based patient pool. If the predicted survival function for the new patient is more similar to that of the EGFR-positive patients than that of the EGFR-negative patients from the similarity-based patient pool, it may be determined that a treatment targeting EGFR can be useful to improve the probability of survival of the new patient.

With the disclosed techniques, a similarity-based patient pool can be identified who not only have similar health conditions to the new patient but also are similar in attributes/conditions that are the most relevant to a clinical prediction. The relevancy of the attributes to the clinical prediction makes it more likely that the medical journeys of the patients in the similarity-based patient pool can provide insights into potential treatments that can improve the prognosis of the new patient. These insights can be backed by the statistics and medical history of a relatively large population of patients. For example, certain biomarkers that are common between the similarity-based patient pool and the new patient can be studied to decide if a targeted treatment may improve the new patient's probability of survival.

### I. Examples of Clinical Prediction and Application

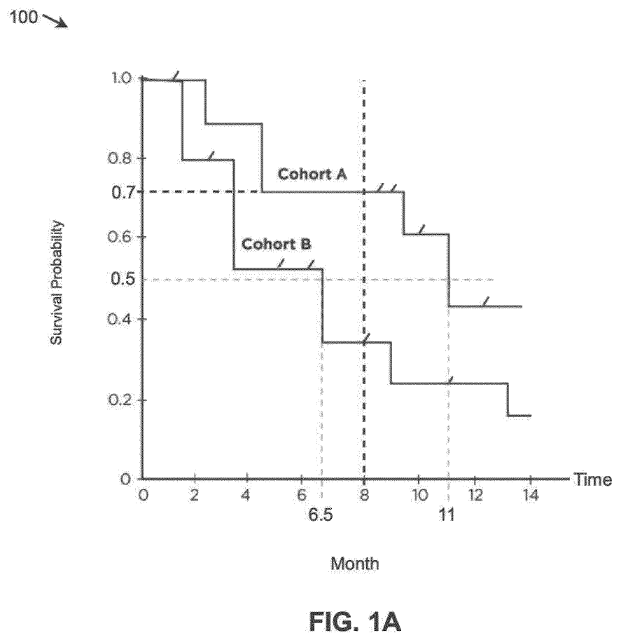

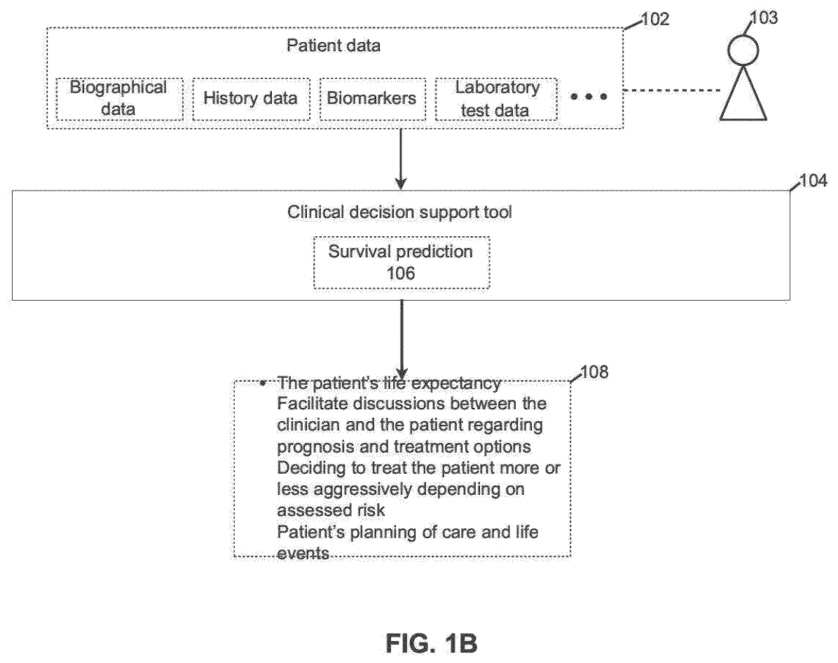

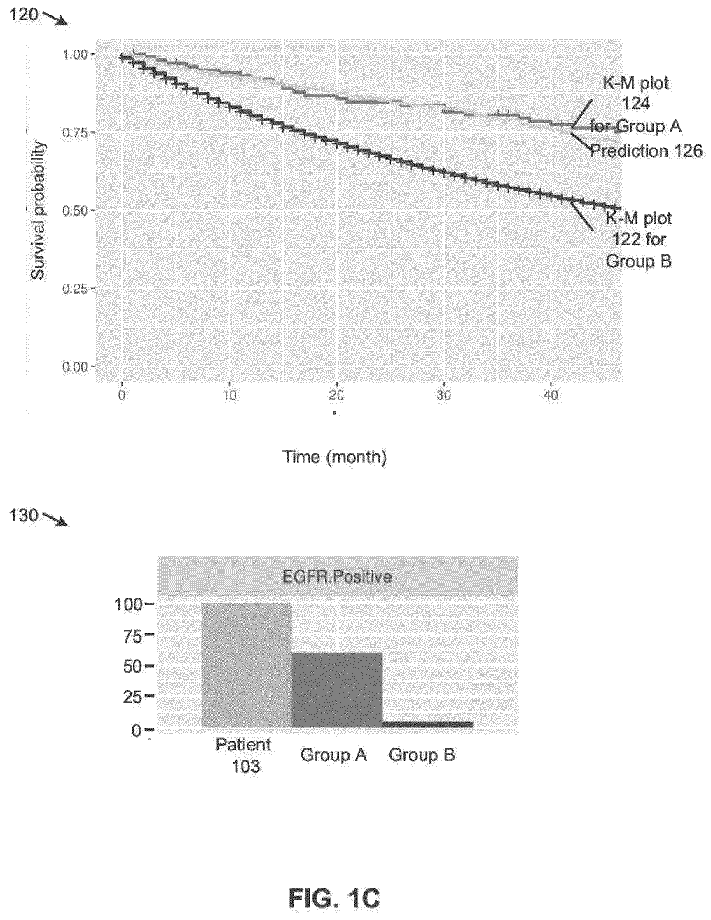

FIG. 1A and FIG. 1B illustrate examples of a clinical prediction that can be provided by embodiments of the present disclosure. FIG. 1A illustrates a mechanism to predict the cumulative survival probability of a patient with respect to time from when a diagnosis of a cancer is made, whereas FIG. 1B illustrates example applications of survival probability prediction. Referring to FIG. 1A, chart 100 illustrates examples of a Kaplan-Meier (K-M) plot, which provides a study of survival statistics among patients having a type of cancer (e.g., lung cancer). The patients may receive a particular treatment. A K-M plot shows the change of cumulative survival probability of a group of patients with respect to time measured from when the patients are diagnosed of a cancer. In a case where the patients receive a treatment, the K-M plot also shows the cumulative survival probability of the patients in response to the treatment. As the time progresses, some patients may die, and the survival probability decreases. Some other patients can be censored (dropped) from the plot due to other events not related to the studied event (e.g. they move to a different state so change hospital). Censored events are represented by diagonal ticks in the K-M plot. The length of each horizontal line represents intervals during which there are no deaths, and the survival estimates at a given point represent the cumulative probability of surviving to that time.

In FIG. 1A, chart 100 includes two K-M plots of the cumulative survival probabilities of different cohorts A and B of patients (e.g., cohorts of patients having different characteristics, receiving different treatments, etc.). From FIG. 1A, the median survival time (first time at which cumulative probability of survival falls below 50%) in cohort A is about 11 months, whereas in cohort B is about 6.5 months. For example, the probability of a patient in cohort A surviving at least 8 months is about 70% (0.7), whereas the probability of a patient in cohort B surviving at least 8 months is about 30% (0.3).

FIG. 1B illustrates example applications of survival prediction for a patient. As shown in FIG. 1B, data 102 of a patient 103 can be input to a clinical decision support tool 104 to generate a survival prediction 106. Data 102 can include different attributes such as, for example, biographical data, history data, biomarkers, laboratory test result data, etc. Clinical decision support tool 104 can generate various information 108 to assist a clinician in administering care/treatment to patient 103 based on survival prediction 106. For example, to facilitate care to patient 103, clinical decision support tool 104 can generate information 108 to indicate, for example, the patient's life expectancy. Information 108 can facilitate discussions between the clinician and patient 103 regarding the patient's prognosis as well as assessment of treatment options, as well as the patient's planning of life events. Two illustrative examples are given as below. If clinical decision support tool 104 predicts that patient 103 has a relatively high probability of survival in 5 years, patient 103 may decide to undergo an aggressive treatment that is more physically demanding and has more serious side effects. But if clinical decision support tool 104 indicates that patient 103 has a relatively low probability of survival in 5 years, patient 103 may decide to forgo the treatment or to undergo an alternative treatment, and plan for care and life events in the remaining life.

Although survival prediction can provide valuable information to the patient and to the clinicians, the survival prediction result by itself may not provide insight into how to improve the prognosis of the patient. For example, a prediction that patient 103 has a certain probability of surviving beyond a certain time point may not provide information about potential treatments to improve the patient's likelihood of survival at that time point.

Clinical decision-making is a complicated task in which clinicians must infer a diagnosis or treatment plan. Clinicians aim to match best treatments based on their education, research and personal experience. They typically operate on a per-patient basis and without digital solutions at hand that could assist them leverage the potential of medical knowledge gained from real-world data (RWD). On the other hand, increasing volume of RWD provides the opportunity to supplement decision making with evidence-based population information. Patient similarity is a fundamental component for researching the most and the least effective treatment on RWD of like individuals with comparable health conditions.

FIG. 1C illustrates an example of a clinical decision-making based on RWD and a clinical prediction result. FIG. 1C illustrates a chart 120 which combines a K-M plot 122 of a first group of patients (labelled “Group A” in FIG. 1C), a K-M plot 124 of a second group of patients (labelled “Group B” in FIG. 1C), and a survival prediction result 126 for patient 103. Survival prediction result 126 for patient 103 can be a function of time in which the predicted cumulative survival probability reduces with time. As shown in FIG. 1C, the predicted cumulative survival probability function 126 of patient 103 is more similar to K-M plot 124 of Group B than to K-M plot 122 of Group A.

Chart 130 illustrates example distribution of positive epidermal growth factor receptor (EGFR) among patient 103, Group A patients (corresponding to K-M plot 124), and Group B patients (corresponding to K-M plot 122). Patient 103 (corresponding to the predicted cumulative survival probability function 126) has positive EGFR, so the bar in chart 130 for patient 103 is at 100%. About 60% of the patients in Group A have a positive EGFR result, while less than 5% of the patients in Group B have a positive EGFR result (both results from chart 130). Note also that the cumulative survival curve 124 is overlapping with curve 126, while curve 122 is substantially lower.

From chart 120 and chart 130, it can be determined that the Group A patients, who have similar cumulative survival curve as patient 103 as evident from the similarity between K-M plot 124 and prediction result 126, have a about 60% EGFR positive rate. In contrast, Group B, whose K-M plot 122 shows much lower survival probabilities than prediction result 126 of patient 103, have only 5% EGFR positive rate. This may suggest that the presence of EGFR may be an important factor in determining survival probability for patient 103. Further study can then be made, such as investigating treatments that target EGFR, based on this observation.

While such an observation is useful and can provide insight into treatment options to improve the probability of survival of patient 103, the observation typically cannot be made from survival probability prediction 106 alone. For example, the prediction result does not identify other patients who have similar survival statistics. The prediction result also does not identify other patients who have similar health conditions as patient 103.

### II. Similarity-Based Patient Pooling Using a Machine Learning Model

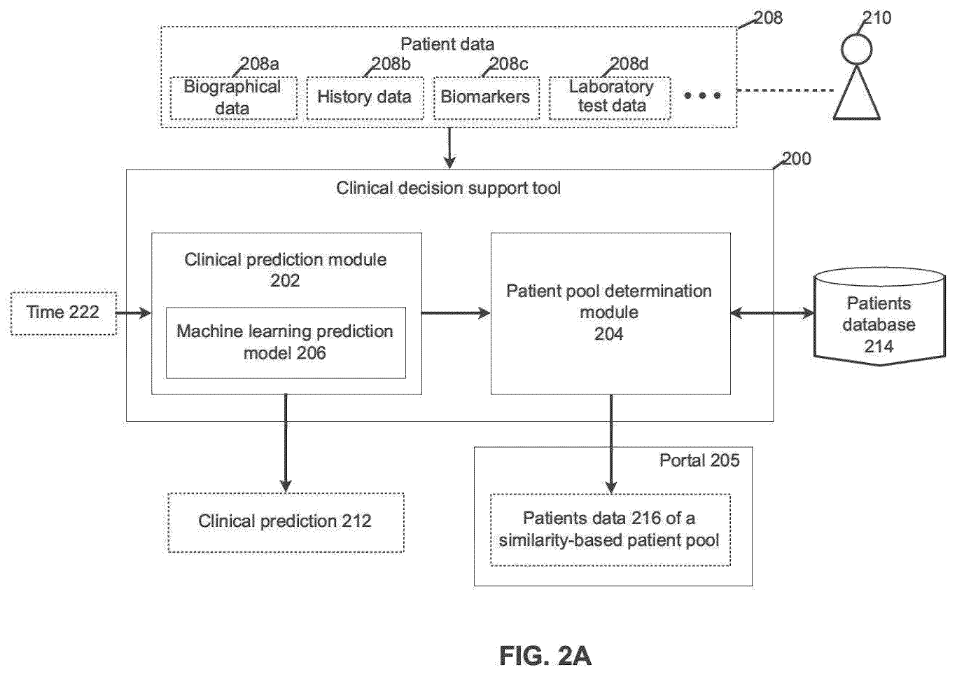

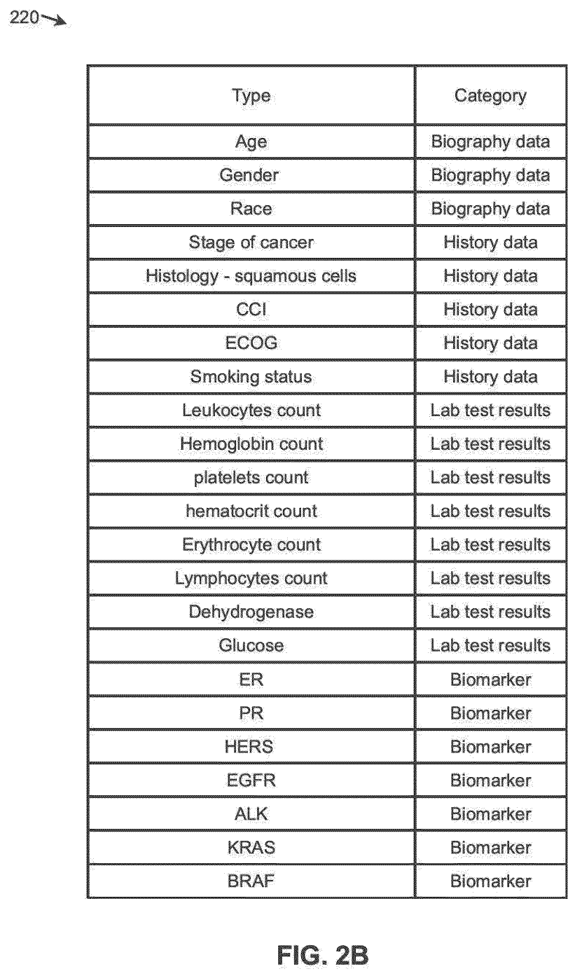

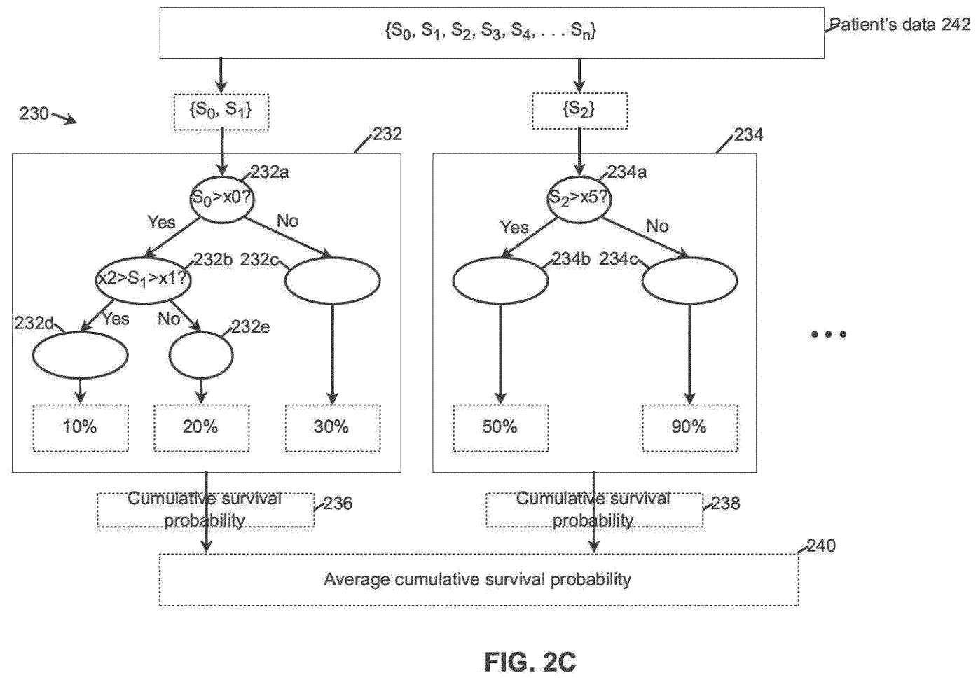

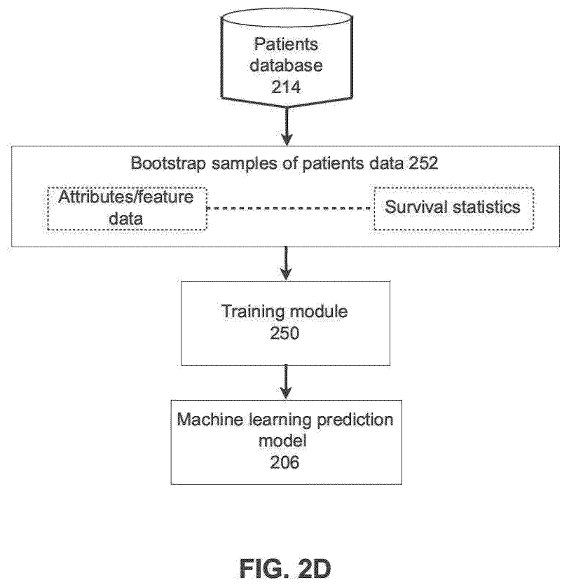

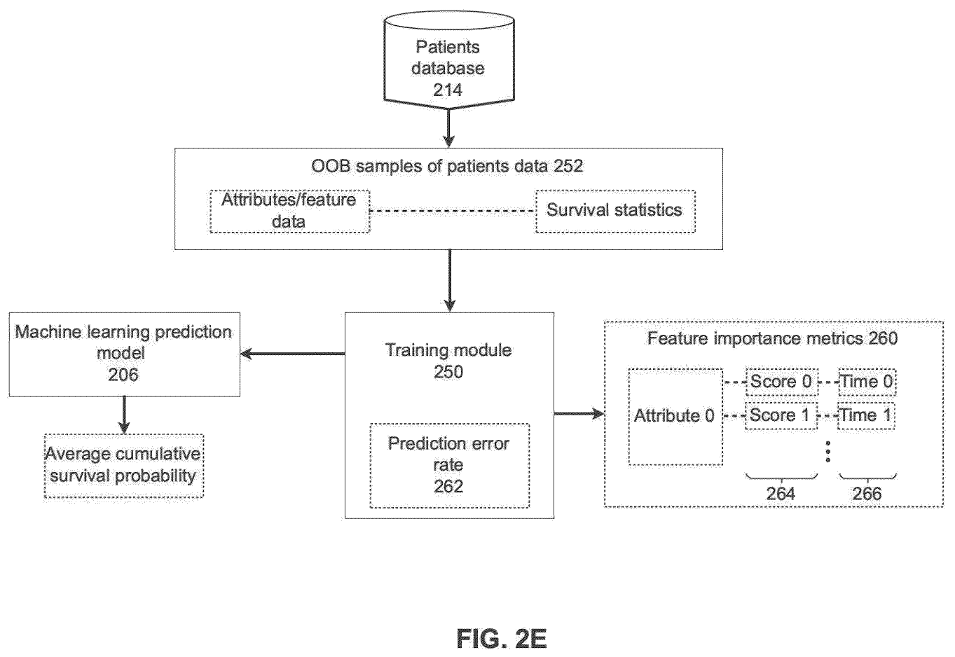

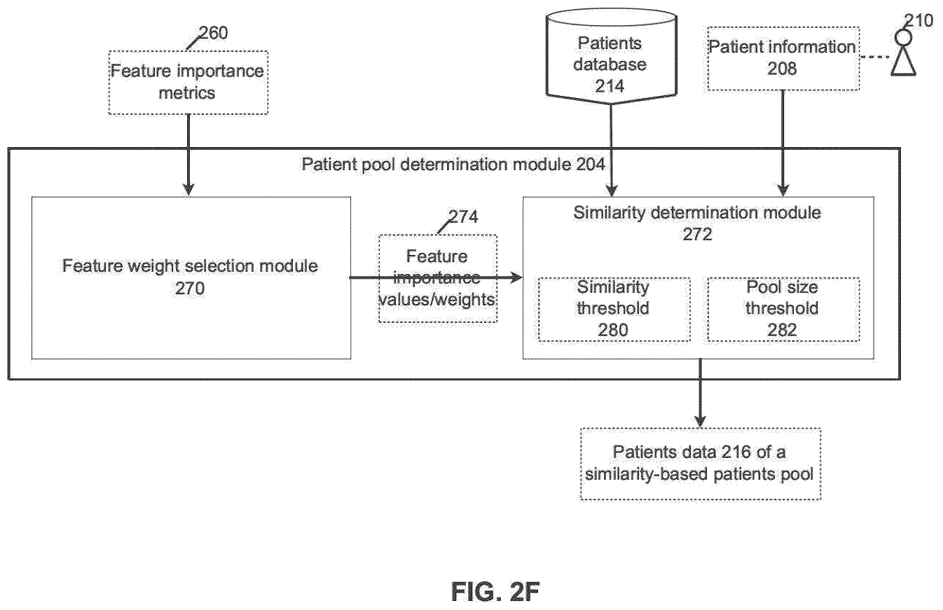

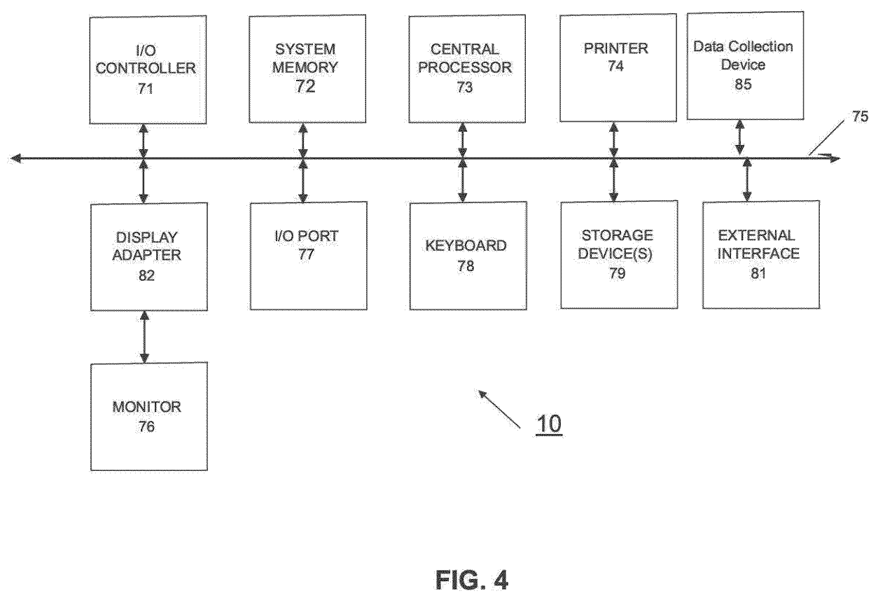

FIG. 2A illustrates an example of a clinical decision support system 200 that performs a clinical prediction for a patient and identifies a similarity-based patient pool (based on patient attributes involved in the clinical prediction). As shown in FIG. 2A, clinical decision support system 200 includes a clinical prediction module 202, a patient pool determination module 204, and a portal 205. Clinical prediction module 202 may include a machine learning prediction model 206. Clinical prediction module 202 can receive patient data 208 corresponding to a plurality of features of a patient 210, and use machine learning prediction model 206 to make a clinical prediction 212 based on patient data 208 for the patient. Patient 210 can be a new patient. The features of patient data 208 may represent various attributes of patient 210 including, for example, biographical data 208a, history data 208b, biomarkers 208c, laboratory test result data 208d, etc. Clinical prediction 212 can include, for example, a probability of survival of the patient. The probability of survival can indicate a likelihood that the patient survives until pre-determined time (e.g., 500 days, 1000 days, 1500 days, etc.) after the patient is diagnosed of a medical condition (e.g., an advanced stage cancer). Clinical decision support system 200 can be a software system executed on a computer system, such as computer system 10 of FIG. 4.

In addition, patient pool determination module 204 can be coupled with a patient database 214 which stores patient data of a set of patients. As to be described below, the patient data of patient database 214 can be used to train machine learning prediction model 206. Patient pool determination module 204 can identify, from patient database 214, a pool of patients having similar attributes as patient 210 and their patient data 216. Patient pool determination module 204 can identify the pool of patients based on these patients being similar to patient 210 in a subset of the attributes that are most relevant to the clinical prediction performed by machine learning prediction model 206. The clinical decision support system can then obtain, from patient database 214, patient data 216 that correspond to the pool of patients. Portal 205 can perform additional processing of patient data 216 (e.g., comparison between patient data 216 of the patient pool and patient data 208 of patient 210).

FIG. 2B illustrates a table 220 which provides examples of attributes included in biographical data 208a, history data 208b, biomarkers 208c, and laboratory test result data 208d. For example, biographical data 208a can include various categories of information, such as age, gender, and race. History data 208b can include various categories of information, such as a diagnosis result including a stage of cancer, histology, Charlson comorbidity index (CCI) which predicts risk of death based on the presence of specific comorbid conditions, Eastern Cooperative Oncology Group (ECOG) value which describes a patient's level of functioning in terms of their ability to care for themselves, daily activity, and physical ability. History data 208b can also include other information, such as a habit of the patient (e.g., whether the patient smokes). Laboratory test results 208c can include different categories of laboratory test results of the patient, such as a leukocytes count, a hemoglobin count, a platelets count, a hematocrit count, an erythrocyte count, a creatinine count, a lymphocytes count, measurements of protein, bilirubin, calcium, sodium, potassium, alkaline phosphatase, carbon dioxide, monocytes, chloride, lactate dehydrogenase, glucose, etc. Biomarker data 208d can include measurements of various biomarkers for different cancer types, such as oestrogen receptor (ER), progesterone receptor (PR), human epidermal growth factor receptor 2 (HER2), epidermal growth factor receptor (EGFR, or HER1) for breast cancer, ALK (anaplastic lymphoma kinase) for lung cancer, KRAS gene for lung and colorectal cancers, BRAF gene for colorectal cancer, etc. It is understood that other attributes of clinical data not shown in FIG. 2B, such as biopsy image data, can also be provided to machine learning prediction model 206 to perform the clinical prediction.

In table 220, each attribute can be represented by a continuous numerical feature, a binary feature (value can be one or zero), or a number of one-hot encoded vectors that indicate one numerical value out of a set of possible categories of the attribute. For example, age can be represented as a continuous numerical feature. As another example, attributes corresponding to the results of testing for biomarker ER can be one-hot encoded. Such attributes can be associated with the following data categories: biomarker result positive, biomarker result negative, biomarker result invalid, and biomarker not tested. The one-hot encoding can generate four features, each corresponding to one of the above categories. For each patient, exactly one of the four features will take the value 1 (the feature corresponding to their category of the attribute), and the other three will take the value 0. This is illustrated in the below table, where examples are given for four patients, each with a different category of the ER biomarker attribute.

TABLE 1
Example attributes of biomarker ER

| | Attribute | One-hot encoded features | | | |
|---|---|---|---|---|---|
| Patient ID | ER biomarker | Positive | Negative | Invalid | Not tested |
| 1 | Positive | 1 | 0 | 0 | 0 |
| 2 | Negative | 0 | 1 | 0 | 0 |
| 3 | Invalid | 0 | 0 | 1 | 0 |
| 4 | Not tested | 0 | 0 | 0 | 1 |

### A. Random Survival Forest

Machine learning prediction model 206 of FIG. 2A can be implemented using various techniques, such as a random survival forest (RSF) model. FIG. 2C illustrates an example of an RSF model 230. As shown in FIG. 2C, random survival forest model 230 can include a plurality of decision trees including, for example, decision trees 232 and 234. Each decision tree can include multiple nodes including a root node (e.g., root node 232a of decision tree 232, root node 234a of decision tree 234), and child nodes (e.g., child nodes 232b, 232c, 232d, and 232e of decision tree 232, child nodes 234b and 234c of decision tree 234). Each parent node that has child nodes (e.g., nodes 232a, 232b and 234a) can be associated with pre-determined classification criteria to classify a patient into one of its child nodes. Child nodes that do not have child nodes are terminal nodes, which include nodes 232d and 232e (of decision tree 232) and nodes 234b and 234c (of decision tree 234). A node survival is calculated at each terminal node of each tree. When used to predict the survival for patient 210, patient 210 is assigned to a terminal node for each tree based on data 208 of patient 210. For example, decision tree 232 can output a cumulative survival probability value 236, whereas decision tree 234 can output a cumulative survival probability value 238. The survival probability for patient 210 can be calculated by averaging the node survivals from each of the terminal nodes to which the patient was assigned. For example, an average survival probability value 240 can be computed based on an average among survival probability values 236, 238, and survival probability values output by other decision trees.

Each decision tree can be assigned to process different subsets of the features. For example, as shown in FIG. 2C, a patient data 242 includes a set of features {S₀, S₁, S₂, S₃, S₄, . . . Sₙ}. Each feature can represent an attribute shown in FIG. 2B, or any other attributes as described herein. Decision tree 232 can be assigned to process features S₀ and S₁, decision tree 234 can be assigned to process feature S₂, while other decision trees can be assigned to process other feature subsets. A parent node in a decision tree can then compare a subset of patient data 242 correspond to one or more of the assigned features against one or more thresholds to classify patient 210 into one of its child nodes. For example, referring to decision tree 232, root node 232a can classify the patient into child node 232b if the patient data of feature S0 exceeds a threshold x0, and into terminal node 232c if otherwise. Child nodes 232b can further classify the parent into one of terminal nodes 232d or 232c based on that patient data of feature S1. Depending on which terminal node the patient is classified into based on features S0 and S1, decision tree 232 can output a cumulative survival probability of 10%, 20%, or 30%. Moreover, decision tree 234 can also output a cumulative survival probability of 50% or 90% depending on which terminal node the patient is classified into based on feature S2.

RSF model 230 of FIG. 2C can be built to determine cumulative survival probability up to a pre-determined time from diagnosis (e.g., 1 year, 3 years, 5 years, etc.). Multiple RSF models can be included in machine learning prediction model 206. Referring back to FIG. 2A, clinical prediction module 202 can receive, as input, time 222 for cumulative survival probability determination. Clinical prediction module 202 can then select the RSF model trained for time 222 to compute the cumulative survival probability up to time 222.

### B. Training Operation

A training operation can be performed to generate each decision tree in a RSF model, the subsets of features assigned to each decision tree, the classification criteria at each parent node of the decision trees, as well as the output value at each terminal node. FIG. 2D illustrates an example of a training operation. The training operation can be performed by a training module 250 which can be part of or external to clinical decision support tool 200 (FIG. 2A). The training operation can be performed using patient data of a large population of patients from patient database 214. As described above, an RSF model can be trained to determine cumulative survival probability up to a pre-determined time from diagnosis. The training data used to train a particular RSF model for determining a cumulative survival probability up to a pre-determined time (e.g., 1, 3, 5, etc. years) from diagnosis can include deaths and censoring up that pre-determined time, and then at the pre-determined time all surviving patients would be censored. In some examples, the training data can include deaths and censoring up to a further time (e.g., 5 years of deaths and censoring data for a RSF model that outputs a cumulative survival probability up to 3 years).

Specifically, patient database 214 can store the attributes of the patients shown in table 220. Training module 250 performs a process of randomly sampling patient data 252 with replacement for the root node of each tree in the RSF model. The process of random sampling with replacement is generally referred to as “bootstrapping”, and because all trees are combined/aggregated to from the random forest, the process is also referred to as “bagging.” Each tree is also assigned a random subset of the features. As part of the training operation, the root node (and each parent node thereafter) can then be split into child nodes in a recursive node-splitting process. In the node-splitting process, a node comprising a subset of patients can be split into two child nodes based on thresholds for the subset of the features. The feature and its threshold at each split are selected to maximize a difference in the survival probabilities between the two child nodes (e.g., based on the log-rank test).

As an example, during the training of decision tree 232 of FIG. 2C, it can be determined that dividing the bootstrap samples of patient data into two groups based on the feature S0 and threshold x0, the difference in the survival probabilities between the two groups can be maximized, such that choosing a different features (e.g., S1) or setting a different threshold for feature S0, would result in a smaller difference between the survival probabilities of the two groups, and a child node 232a can be generated. The process can then be repeated on child node 232a to generate additional child nodes until, for example, a threshold minimum number of patients is reached in a particular child node. Once the splitting process has been stopped, all childless nodes can become terminal nodes. For example, in at least one of terminal nodes 232d, and 232e, the number of patients reaches the threshold minimum number, and therefore the root-splitting operation stops at these nodes. The output at each of these terminal nodes can be calculated from the outcome data of the patients classified into that terminal node. The training operation can be repeated to generate the decision trees for outputting the survival probabilities at different times, such that RSF model 230 can output a survival function that predicts the survival probability of a patient at different times.

Referring to FIG. 2E, training module 250 can also determine feature importance metrics 260 associated with the machine learning prediction model 206. Feature importance metrics 260 can define the relevance of each feature by investigating its effect on the error of the machine learning prediction model 206. Feature importance metrics 260 can be determined for survival probability up to a pre-determined time (e.g., 3 years), and different feature importance metrics 260 can be determined for survival probability prediction up to different pre-determined times (e.g., 3 years, 5 years, 7 years, etc.) by machine learning prediction model 206.

In one example, to determine feature importance metrics 260, training module 250 can obtain a set of out-of-bag (OOB) samples of patient data 252 from patient database 214. The OOB samples for each tree can include samples of patient data not included in the bootstrap samples for that tree in FIG. 2D. For those samples, the values for a feature can be permuted, and a prediction error rate 262 for each decision tree from processing the OOB samples with the permuted values of that feature can be obtained. A raw importance score 264 can be computed for that feature based on, for example, averaging a difference in the prediction error rate outputs by each decision tree. The process can be repeated for each feature to compute a separate raw importance score 264 for each feature. A high raw importance score can indicate that the feature is more relevant to survival prediction, whereas a low raw importance score can indicate that the feature is less relevant. At the end of the training operations, the features can be ranked based on their importance scores, with more relevant features ranked higher.

In one example, training module 250 can compute prediction error rate 262 based on computing a concordant index (c-index). The concordant index can be computed for the OOB samples based on performing a pairwise comparison of the model's estimate of the cumulative hazard function (CHF) and the actual time of death between patients in the OOB samples. For each pair of patients, if the relative survival probabilities of the pair, at a given time point, matches the time order of death of the pair, then the pair is concordant, otherwise the pair is disconcordant. For example, if the CHF estimate of a first patient of the pair is higher than that of a second patient of the pair, and the first patient died before the second patient, then the pair is concordant. Otherwise, the pair is disconcordant. The c-index can be computed based on the following equations:

$$C - index = \frac{number of concordant pairs}{\begin{matrix}number of concordant pairs + \\ number of discordant pairs\end{matrix}} \qquad ( Equation 1 )$$

The prediction error rate can then be computed as an inverse of the C-index. As the survival probability may change with time, the prediction error rate, as well as the resulting raw importance score, may also change with time. Therefore, as shown in FIG. 2E, feature importance metrics 260 may include different raw importance scores 264 for different times 266.

### C. Similarity-Based Patient Pooling

Based on feature importance metrics 260, as well as patient data 208 of patient 210, patient pool determination module 204 can identify, from patient database 214, a pool of patients having similar attributes as patient 210 and their patient data 216. FIG. 2F illustrates example internal components of patient pool determination module 204 and their operations. As shown in FIG. 2F, patient pool determination module 204 includes feature weight selection module 270 and similarity determination module 272.

Feature weight selection module 270 can rank the features by the feature importance values 260 and select the x features with the highest feature importance values (x can be a predetermined number, e.g. 20, or based on a rule, e.g. all features whose importance value is greater than the average importance value across all features). The set of the top x features can be denoted E. Feature weight selection module 270 can then fit an RSF using only the features in E, and recalculate the feature importance values for these features from this new RSF. These new raw feature importance values, denoted wₖ for feature k, were scaled according to the following equation:

$${\sum }_{k ∈ E} {w}_{k} = 1 ; {w}_{k} > 0 for k ∈ E \qquad ( Equation 2 )$$

The scaled feature importance values, wₖ, are then used as weights in similarity determination module 272.

Similarity determination module 272 can then identify patients in patient database 214 who are similar to patient 210 based on scaled feature importance values/weights 274. Similarity determination module 272 can determine a weighted aggregated similarity s(xᵢ, xⱼ) between two patients, xᵢ and xⱼ, based on the following equation:

$$s ( {x}_{i} , {x}_{j} ) = {\sum }_{k} {w}_{k} {s}_{ijk} \qquad ( Equation 3 )$$

In Equation 3, sᵢⱼₖ represents a degree of similarity in a feature k between patients xᵢ and xⱼ, whereas wₖ is a scaled feature importance value 274. A more important feature can have the degree of similarity associated with a larger weight. In a case where feature k is represented by a binary value or a one-hot encoded vector, the degree of similarity sᵢⱼₖ can be a one if feature k for both patients is one, or that the one-hot encoded vectors match completely, otherwise sᵢⱼₖ can take on a value of zero. Moreover, in a case where feature k takes on a numerical value from a range Rₖ, the degree of similarity sᵢⱼₖ can be computed based on the following equation:

$${s}_{ijk} = 1 - \frac{\vert {x}_{ik} - {x}_{jk} \vert}{{R}_{k}} \qquad ( Equation 4 )$$

Similarity determination module 272 can compute the weighted aggregated similarity s(xᵢ, xⱼ) between patient 210 and the patients represented in patient database 214 using Equation 3, and select a similarity-based patient pool based on the weighted aggregated similarities. The similarity-based patient pool may be considered to not only have similar health conditions as the patient, but also being similar to the patient in the features that are most relevant to the clinical prediction. In one example, similarity determination module 272 can select the similarity-based patient pool based on their degrees of similarity to patient 210, computed according to Equations 3 or 4, exceeding a similarity threshold 280. In another example, similarity determination module 272 can select a pre-determined number of patients, defined based on pool size threshold 282, having the highest degree of similarity to patient 210 to be part of the similarity-based patient pool.

Similarity determination module 272 can then obtain the attributes and the medical outcomes of the similarity-based patient pool, and output them as part of patient data 216, to facilitate a clinical decision for patient 210. For example, referring back to FIG. 1C, similarity determination module 272 can identify a similarity-based patient pool for which K-M curve 124 can be generated, as well as patient data 216. Portal 205 can perform a comparison among patient data 216 of the patient pool, patient data 208, and the patient data of the training set of patients in patient database 214 for each feature present in both sets of data, and output the comparison result. From the comparison, a clinician may determine that EGFR positivity of the patient pool is much higher than that of the training set of patients, and may perform further investigation into EFGR (e.g., a treatment targeted at EFGR) to improve the survival rate of the patient.

### III. Method

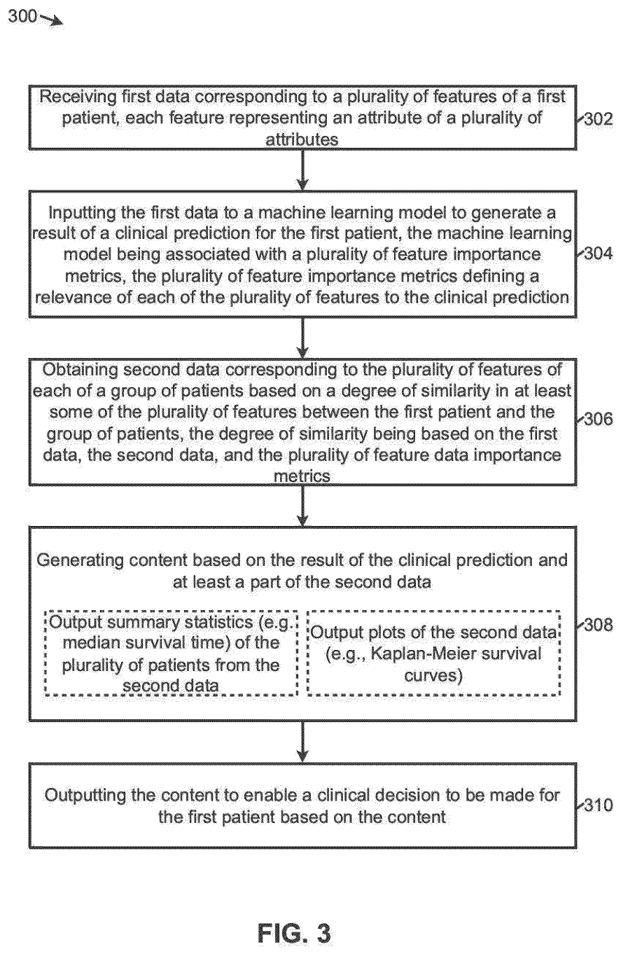

FIG. 3 illustrates an example of a method 300 of facilitating a clinical decision. Method 300 can be performed by, for example, clinical decision support tool 200 of FIG. 2A.

In step 302, the clinical decision support tool can receive first data corresponding to a plurality of features of a first patient (e.g., a new patient), with each feature representing an attribute of a plurality of attributes. The first data can be input via a computer interface, such as portal 205, or directly from a patients database, such as patients database 214. The first patient can be a new patient, such as patient 210.

Referring to FIG. 2A, first data can include patient data 208 corresponding to the attributes of the new patient. The attributes can include various biographical information, such as age and gender of the patient. Each attribute can be represented as one or more features, each feature can be represented as a vector for input into the machine learning model. The attributes may also include a history of the patient (e.g., which treatment(s) the patient has received), a habit of the patient (e.g., whether the patient smokes), categories of laboratory test results of the patient (e.g. leukocytes count, a hemoglobin count, a platelets count, a hematocrit count, an erythrocyte count, a creatinine count, a lymphocytes count, measurements of protein, bilirubin, calcium, sodium, potassium, glucose). The attributes may also indicate measurements of various biomarkers for different cancer types, such as oestrogen receptor (ER), progesterone receptor (PR), human epidermal growth factor receptor 2 (HER2), epidermal growth factor receptor (EGFR, or HER1) for breast cancer, ALK (anaplastic lymphoma kinase) for lung cancer, KRAS gene for lung and colorectal cancers, BRAF gene for colorectal cancer, etc.

In step 304, the clinical decision support tool can input the first data to a machine learning model to generate a result of a clinical prediction for the first patient, the machine learning model being associated with a plurality of feature importance metrics, the plurality of feature importance metrics defining a relevance of each of the plurality of features to the clinical prediction.

Referring to FIG. 2A, the clinical decision support tool can include machine learning prediction model 206 to make a clinical prediction 212 based on the data for the patient. Machine learning model 206 may include RSF model 230 of FIG. 2C that can output, as the clinical prediction, a predicted survival function, based on the data for the patient. Clinical prediction 212 can include, for example, a probability of survival of the patient. The probability of survival can indicate a likelihood that the patient survives until a pre-determined time (e.g., 500 days, 1000 days, 1500 days, etc.) after the patient is diagnosed of a medical condition (e.g., an advanced stage cancer). As described above, in some examples, machine learning prediction model 206 may include multiple RSF models configured to predict a probability of survival up to different pre-determined times. One of the RSF models can be selected based on an input time predict the probability of survival up to the input time.

In addition, the machine learning prediction model 206 is also associated with a plurality of feature importance metrics, such as feature importance metrics 260. Referring to FIG. 2E, feature importance metrics 260 can define the relevance of each feature by investigating its effect on the error of the machine learning prediction model 206 and can be determined by training module 250 based on a set of out-of-bag (OOB) samples of patient data 252 from patient database 214. The OOB samples can include samples of patient data not involved in the bagging process used to construct RSF model 230. For those samples, the values for a feature can be permuted, and a prediction error rate for each decision tree from processing the OOB samples with the permuted values of that feature can be obtained. The prediction error rate can be computed based on a concordant index (c-index) based on Equation 1 above. A raw importance score can be computed for that feature based on, for example, averaging a difference in the prediction error rate outputs by each decision tree. The process can be repeated for each feature to compute a separate raw importance score for each feature. A high raw importance score can indicate that the feature is more relevant to survival prediction, whereas a low raw importance score can indicate that the feature is less relevant. At the end of the training operations, the features can be ranked based on their importance scores, with more relevant features ranked higher.

In step 306, the clinical decision support tool can obtain second data corresponding to the plurality of features of each of a group of patients based on a degree of similarity in at least some of the plurality of features between the first patient and the group of patients, the degree of similarity being based on the first data, the second data, and the plurality of feature importance metrics.

Specifically, the second data can be obtained by patient pool determination module 204 of clinical decision support tool 200, which includes feature weight selection module 270 and similarity determination module 272. Referring to FIG. 2F, feature weight selection module 270 can rank the features by the feature importance values 260 and select the x features with the highest feature importance values (x can be a predetermined number, e.g. 20, or based on a rule, e.g. all features whose importance value is greater than the average importance value across all features). The set of the top x features can be denoted E. Feature weight selection module 270 can then fit an RSF using only the features in E, and recalculate the feature importance values for these features from this new RSF. Degrees of similarity between the first patient and other patients can be computed based on Equations 3-4 above. In the first example, the patients can be selected based on their similarity to the new patient exceeding a threshold. In the second example, patients in the database are ranked according to their similarity to the first patient, and a pre-determined number of patients with the highest rank are selected.

In step 308, the clinical decision support tool can generate content based on the result of the clinical prediction and at least a part of the second data.

Specifically, in some examples, the content may include output summary statistics (e.g., median survival time) of the patient pool (group of patient), K-M curves of the patient pool, etc. In some examples, a comparison among patient data of the group of patients, the first patient, and the training set of patients (e.g., patients represented in the training data set that trains the machine learning model) can be made to generate a comparison result.

In step 310, the clinical decision support tool can output the content to enable a clinical decision to be made for the first patient based on the content. For example, referring back to FIG. 1C, the content may indicate that the EGFR positivity of the patient pool is much higher than that of the training set of patients, and may perform further investigation into EFGR (e.g., a treatment targeted at EFGR) to improve the survival rate of the patient.

### IV. Computer System

Any of the computer systems mentioned herein may utilize any suitable number of subsystems. Examples of such subsystems are shown in FIG. 4 in computer system 10. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices. In some embodiments, a cloud infrastructure (e.g., Amazon Web Services), a graphical processing unit (GPU), etc., can be used to implement the disclosed techniques.

The subsystems shown in FIG. 4 are interconnected via a system bus 75. Additional subsystems such as a printer 74, keyboard 78, storage device(s) 79, monitor 76, which is coupled to display adapter 82, and others are shown. Peripherals and input/output (I/O) devices, which couple to I/O controller 71, can be connected to the computer system by any number of means known in the art such as input/output (I/O) port 77 (e.g., USB, FireWire®). For example, I/O port 77 or external interface 81 (e.g. Ethernet, Wi-Fi, etc.) can be used to connect computer system 10 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A computer system can include a plurality of the same components or subsystems, e.g., connected together by external interface 81 or by an internal interface. In some embodiments, computer systems, subsystem, or apparatuses can communicate over a network. In such instances, one computer can be considered a client and another computer a server, where each can be part of a same computer system. A client and a server can each include multiple systems, subsystems, or components.

Aspects of embodiments can be implemented in the form of control logic using hardware (e.g. an application specific integrated circuit or field programmable gate array) and/or using computer software with a generally programmable processor in a modular or integrated manner. As used herein, a processor includes a single-core processor, multi-core processor on a same integrated chip, or multiple processing units on a single circuit board or networked. Based on the disclosure and teachings provided herein, a person of ordinary skill in the art will know and appreciate other ways and/or methods to implement embodiments of the present invention using hardware and a combination of hardware and software.

Any of the software components or functions described in this application may be implemented as software code to be executed by a processor using any suitable computer language such as, for example, Java, C, C++, C #, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk), flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

### V. EXAMPLES

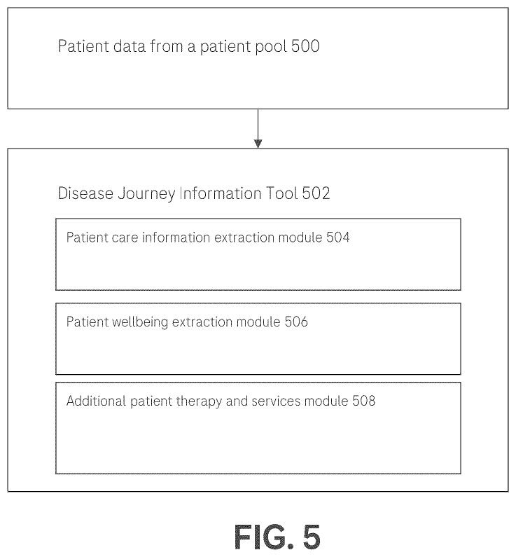

FIG. 5 illustrates one example of how the patient data from a patient pool 500 can be used. Constructing the patient data from the patient pool involves cohort building, any suitable type of cohort building method can be used, such as a similarity-based patient pooling method as described herein. For example, the methods of generating patient data from a similarity based patient pool as described in connection with FIGS. 2A and 2F can be used in the examples described herein. Other methods of cohort building can be suitable too.

As shown in FIG. 5, the patient data from the patient pool 500 can be accessed, processed, and/or used by a disease journey information tool 502 to automatically extract useful data regarding a patient's disease journey from patient electronic health records and/or other patient data bases. In some embodiments, the disease journey information tool 502 can include a patient care information extraction module 504, a patient wellbeing extraction module 506, and an additional patient therapy and services module 508. Various embodiments of the disease journey information tool 502 can include any combination of one or more of the modules described herein.

In some embodiments, the patient care information extraction module 504 includes an algorithm to extract information on the sequence of how patients are cared for in the cohort. This extracted information can be displayed to a user to facilitate and enable the user to learn from disease journeys in a specific cohort. This extracted information can also be utilized in a risk factor analysis in the cohort.

In some embodiments, the patient wellbeing extraction module 506 can include an algorithm to extract information on patients' wellbeing from the patient data. For example, some measures of patients' wellbeing include, for example, patient reported outcomes or experiences, such as reported symptoms, disability, aspects of well-being, health perceptions, and the like. In some embodiments, a user can be provided with a more tailored display that can be used to view the patients' wellbeing an both an individual level and as groups of patients within the cohort. For example, a user can be provided with population statistics on how patients in the identified cohort reported on their treatments.

In some embodiments, the additional patient therapy and services module 508 can include an algorithm to extract information on non-medical additional services (i.e., non-pharmaceutical and non-drug services), such as rehabilitation, psychological therapy, physical therapy, and occupational therapy, for example. In some embodiments, this extracted information can be used to determine which additional non-medical interventions benefited a specific patient cohort, e.g. rehabilitation clinic, psychological therapy, physical therapy, and/or occupational therapy.

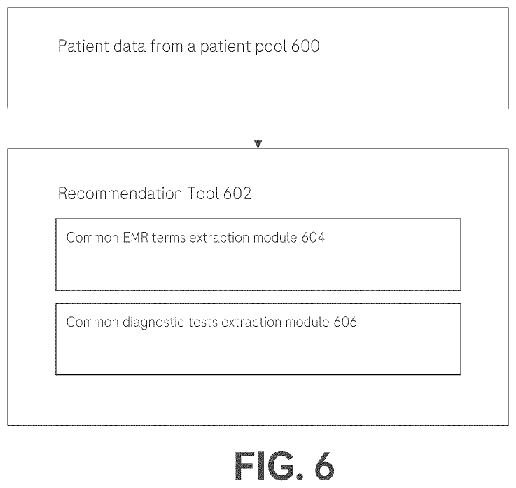

FIG. 6 illustrates another example of how the patient data from a patient pool 600 can be used. As shown in FIG. 6, the patient data from the patient pool 600 can be accessed, processed, and used by a recommendation tool 602 to suggest common terms for entry into data fields of an electronic form or record. In some embodiments, the recommendation tool 602 can include a common electronic medical records (EMR) terms extraction module 604 and a common diagnostic tests extraction module 606. Various embodiments of the recommendation tool 602 can include any combination of one or more of the modules described herein.

In some embodiments, the common EMR terms extraction module 604 can include an algorithm to extract common terms that are used for data fields in an EMR system and then use these extracted common terms as recommendations to users that are filling out an EMR. For example, in some embodiments one or more of the data fields in an EMR that a user is filling out can be filled out automatically with preselected text fields according to the highest frequency of the extracted common terms in the cohort. In some embodiments, the user may instead be provided by a sorted list of common terms when the text field is selected, where the list is sorted based on the frequency the term is found in the cohort. In some embodiments, the common EMR terms can be extracted from the EMRs from the cohort patient pool. In other embodiments, the common EMR terms can be extracted from a broader EMR dataset formed from a larger patient pool. In some embodiments, the common EMR terms can be extracted from one or more EMR datasets.

In some embodiments, the common diagnostic tests extraction module 606 can include an algorithm to extract common diagnostic tests for a diagnostic test recommendation system. In some embodiments, the dataset used to extract the common diagnostic tests can be limited to the data from the cohort patient pool. In some embodiments, the methods of generating patient data from a similarity based patient pool as described in connection with FIGS. 2A and 2F can be used to build a cohort that has similar characteristics as a particular patient. The recommendation system identifies the diagnostic tests performed in this cohort. The extracted information can be used by the system to recommend these tests to be considered for the particular patient.

In some embodiments, the algorithm used by the data extraction modules described herein can be, but is not limited to, a process mining algorithm, a deep learning algorithm, and sequence alignment methods.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective steps or a respective group of steps. Although presented as numbered steps, steps of methods herein can be performed at a same time or in a different order. Additionally, portions of these steps may be used with portions of other steps from other methods. Also, all or portions of a step may be optional. Additionally, any of the steps of any of the methods can be performed with modules, units, circuits, or other means for performing these steps.

The specific details of particular embodiments may be combined in any suitable manner without departing from the spirit and scope of embodiments of the invention. However, other embodiments of the invention may be directed to specific embodiments relating to each individual aspect, or specific combinations of these individual aspects.

The above description of example embodiments of the invention has been presented for the purposes of illustration and description. It is not intended to be exhaustive or to limit the invention to the precise form described, and many modifications and variations are possible in light of the teaching above.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover reference to a “first” or a “second” component does not limit the referenced component to a particular location unless expressly stated.

All patents, patent applications, publications, and descriptions mentioned herein are incorporated by reference in their entirety for all purposes. None is admitted to be prior art.

What is claimed is:

1. A computer-implemented method of facilitating a clinical decision, comprising:
receiving first data corresponding to a plurality of features of a first patient, each feature representing an attribute of a plurality of attributes;
inputting the first data to a machine learning model to generate a result of a clinical prediction for the first patient, the machine learning model being associated with a plurality of feature importance metrics, the plurality of feature importance metrics defining a relevance of each of the plurality of features to the clinical prediction;
obtaining second data corresponding to the plurality of features of each of a group of patients based on a degree of similarity in at least some of the plurality of features between the first patient and the group of patients, the degree of similarity being based on the first data, the second data, and the plurality of feature data importance metrics;
generating content based on the result of the clinical prediction and at least a part of the second data; and
outputting the content to enable a clinical decision to be made for the first patient based on the content.

2. The method of claim 1, wherein the plurality of attributes comprises at least one of: biography data of a patient, results of one or more laboratory tests of the first patient, biopsy image data of the first patient, molecular biomarkers of the first patient, a tumor site of the first patient, or a tumor stage of the first patient.

3. The method of claim 1, wherein the plurality of attributes comprise one or more attributes representing measurements of biomarkers for different cancer types.

4. The method of claim 1, wherein the clinical prediction comprises at least one of: a probability of survival of the first patient at a pre-determined time from when the first patient is diagnosed of having a tumor, a survival time of the first patient from when the first patient is diagnosed of having the tumor, or an outcome of receiving a treatment.

5. The method of claim 4, wherein the machine learning model comprises a random forest survival model, the random forest survival model comprising a f decision trees each configured to process a subset of the first subset of the data to generate a cumulative survival probability; and
wherein the survival rate of the patient at the pre-determined time is determined based on an average of the cumulative survival probabilities output by the plurality of decision trees.

6. The method of claim 1, wherein the group of patients is a first group of patients;
wherein the first group of patients is selected from a second group of patients; and
wherein the machine learning model is trained based on patient data of the second group of patients.

7. The method of claim 6, further comprising:
ranking the plurality of features based on the relevance of each feature of the plurality of features to the clinical prediction;
determining a subset of the plurality of features based on the ranking; and
determining the first group of patients based on the degree of similarity in the subset of the plurality of features between the first patient and the first group of patients.

8. The method of claim 7, wherein the first group of patients is selected from the second group of patients based on the degree of similarity in the subset of the plurality of features between the first patient and the first group of patients exceeding a threshold.

9. The method of claim 7, wherein the first group of patients is selected from the second group of patients based on selecting a threshold number of patients having the highest degree of similarity in the subset of the plurality of features with the first patient.

10. The method of claim 1, further comprising:
computing a weighted aggregated degree of similarity based on summing a scaled degree of similarity in each feature of the at least some of the plurality of features, each degree of similarity being scaled by a weight based on the relevance of the feature; and
identifying the group of patients based on the weighted aggregated degree of similarities between the first patient and each of the group of patients.

11. The method of claim 1, wherein the feature importance metric of a feature is determined based on a relationship between errors of the results of clinical prediction generated by the machine learning model for a second patient of the first group of patients;
wherein the results of clinical prediction are generated from a plurality of values of the feature of the second patient; and
wherein the errors are computed based on comparing the results of the clinical prediction and an actual clinical outcome of the second patient.

12. The method of claim 6, wherein the content comprises at least one of: a median survival time of the first group of patients, or a Kaplan-Meier survival curve of the first group of patients.

13. The method of claim 6, wherein the content includes values of one or more of the first subset of the plurality of features of the first patient, the first group of patients, and the second group of patients.

14. A computer product comprising a computer readable medium storing a plurality of instructions for controlling a computer system to perform an operation of any of the methods above.

15. A system comprising:
one or more processors programmed and configured to:
receive first data corresponding to a plurality of features of a first patient, each feature representing an attribute of a plurality of attributes;
input the first data to a machine learning model to generate a result of a clinical prediction for the first patient, the machine learning model being associated with a plurality of feature importance metrics, the plurality of feature importance metrics defining a relevance of each of the plurality of features to the clinical prediction;
obtain second data corresponding to the plurality of features of each of a group of patients based on a degree of similarity in at least some of the plurality of features between the first patient and the group of patients, the degree of similarity being based on the first data, the second data, and the plurality of feature data importance metrics;
generate content based on the result of the clinical prediction and at least a part of the second data; and
output the content to enable a clinical decision to be made for the first patient based on the content.

16. The system of claim 15, wherein the plurality of attributes comprises at least one of: biography data of a patient, results of one or more laboratory tests of the first patient, biopsy image data of the first patient, molecular biomarkers of the first patient, a tumor site of the first patient, or a tumor stage of the first patient.

17. The system of claim 15, wherein the plurality of attributes comprise one or more attributes representing measurements of biomarkers for different cancer types.

18. The system of claim 15, wherein the clinical prediction comprises at least one of: a probability of survival of the first patient at a pre-determined time from when the first patient is diagnosed of having a tumor, a survival time of the first patient from when the first patient is diagnosed of having the tumor, or an outcome of receiving a treatment.

19. The system of claim 18, wherein the machine learning model comprises a random forest survival model, the random forest survival model comprising a f decision trees each configured to process a subset of the first subset of the data to generate a cumulative survival probability; and
wherein the survival rate of the patient at the pre-determined time is determined based on an average of the cumulative survival probabilities output by the plurality of decision trees.

20. The system of claim 15, wherein the group of patients is a first group of patients;
wherein the first group of patients is selected from a second group of patients; and
wherein the machine learning model is trained based on patient data of the second group of patients.
